(19) United States
(12) Patent Application Publication
(10) Pub. No.: US 2007/0032792 A1
(43) Pub. Date: Feb. 8, 2007

(54) Surgical suture anchor element

(76) Inventors: Philippe Collin, Clayes (FR); Sascha Berberich, Tuttlingen (DE)

Correspondence Address: ST. ONGE STEWARD JOHNSTON & REENS, LLC, 986 BEDFORD STREET, STAMFORD, CT, 06905-5619, US

(21) Appl. No.: 11/499,272

(22) Filed: Aug. 4, 2006

(30) Foreign Application Priority Data
Aug. 5, 2005 (DE) 10 2005 039 080.3

(51) Int. Cl. A61B 17/58

(52) U.S. Cl. 606/72

(57) ABSTRACT

A surgical suture anchor element serves for anchoring at least one suture in a bore. An elongated body has a central bore for receiving a tool. The central bore is surrounded by a jacket of considerable substance. For receiving the suture, slits or channels or eyelet members are alternatively provided in or at the jacket.

### BACKGROUND OF THE INVENTION

The invention relates to a surgical suture anchor element for introduction into a bone, with at least one suture for fixing a tendon, a ligament or soft tissue, with at least one opening for receiving the at least one suture, and with a central bore for receiving a tool for introducing the anchor element.

Such anchor elements are known from U.S. Pat. No. 6,139,565, for example.

These anchor elements, also referred to as suture anchors, are used in surgery for fixing a torn tendon, a ligament or other tissue back onto the bone with the aid of a suture. They can also be used for fixation of soft tissue, for example in labrum refixation.

As regards the design of the anchor elements, the primary aim is to ensure that a suture for fixing the torn tendon is held securely by the introduced anchor element. This assumes similar considerations regarding the design of a suture holder in the anchor element.

One of the main fields of application for such anchor elements is the fixing of torn tendons in the shoulder region.

An example of such an anchor element is shown in FIGS. 8A to 8F of aforementioned U.S. Pat. No. 6,139,565.

The anchor element shown there comprises an elongate, cylindrical body with a central bore. This central bore is designed as a channel with a square cross section routed axially through the anchor element and serves to receive the rotary tool used for turning the anchor element into the bone.

On a proximal portion of the anchor element, four webs are arranged in a cross formation around the opening of the channel, and a suture eyelet is cut out in each of these webs.

Since the suture eyelets are intended to receive and hold the suture, it is considered disadvantageous that the suture eyelets are cut out in proximally projecting webs. The webs as such already constitute areas of material weakness in the body of the anchor element, and they are further weakened by the suture eyelets.

The suture is threaded into the narrow suture eyelets of the anchor element.

After the anchor element has been fitted in place, the rotary tool is removed. The tendon is then fixed using the ends of the suture protruding from the bore. Considerable forces act on the suture eyelet both during fixation and also in the postoperative phase. With suture eyelets formed in narrow webs of thin material, there is a danger of the suture coming loose from the anchor element as a result of the material breaking. Narrow webs constitute points of minimal surface contact with the suture and, as a result of movements, promote fraying of the suture.

Since anchor elements made of absorbable materials are also used in modern medicine, that is to say materials that can gradually be replaced by endogenous bone substance, a problem arises due to the materially thin design of the suture eyelet.

This problem entails the danger of the suture eyelet being torn off or broken off, which may lead to serious complications especially in the later stages during the healing process.

It is therefore an object of the present invention to make available an anchor element of the type mentioned in the introduction which provides improved stability for a suture holder.

### SUMMARY OF THE INVENTION

According to the invention, the object is achieved, in one alternative, by an anchor element having an elongated body, said body having a central bore for receiving a tool for introducing said anchor element into said bone, said body having a proximal end section surrounding said central bore, said end section being designed as a jacket of considerable substance, wherein at least one slit is provided in said jacket of considerable substance, said slit opens laterally to a radial outer face of said body, said at least one slit extending in a circumferential direction within said jacket of considerable substance, and said at least one suture is received within said at least one slit.

Within the meaning of the present invention, a “central bore” is to be understood both as a blind hole and also as a continuous channel.

The expression “jacket of considerable substance” is to be understood as a portion of the anchor element that surrounds the central bore in the proximal area. This portion is unweakened by material reduction and is therefore particularly well suited for receiving suture guides. This portion has a thick wall of material.

A suture holder in the form of a slit of this design offers the suture a correspondingly large contact surface. This contact surface extends about part of the circumference of the jacket of considerable substance of the anchor element. In this way, the forces acting on the suture are passed on by it to a solid portion of the anchor element.

The greater the part of the circumference in which the suture is received in the anchor element, the greater also is the contact surface of the suture. In this way, the forces acting on the suture are conveyed uniformly to the anchor element across a greater surface area, and locally concentrated forces are avoided. This protects the suture holder from being torn out or broken off, and it also protects the suture during relative movements between the suture and the anchored anchor element.

This embodiment also has the advantage that the suture holder and the course of a central bore in the anchor element can be kept spatially separate from one another.

This allows the depth of the central bore to be adapted to different designs of rotary tools, independently of the design of the suture receiving part. In this way, an optimal depth of introduction of a tool into the anchor element is ensured.

In addition, insertion of the suture is made easier, since the latter needs only to be pushed into the slit. This avoids the suture and the rotary tool getting in each other's way.

In one embodiment, the at least one slit has flanks that extend in parallel.

This measure has the advantage that the slit, for example in an anchor element made of metal, can be easily produced by milling. In the case of absorbable materials, such an anchor element can be easily produced in an injection-molding operation.

In another embodiment, the at least one slit has flanks that diverge.

This measure has the advantage that the slit is widened laterally toward the radial outer surface by the diverging flanks. This widened slit makes inserting the suture easier. In this case, the flanks can be formed in the anchor element in such a way that, by pulling the received suture in the proximal direction, said suture easily slides radially inward into the slit and remains there as long as the suture is held taut.

In another embodiment, the flanks are beveled at their edges.

This measure has the advantage that beveling the edges prevents the suture from becoming damaged by fraying or from being severed.

In another embodiment, the slits are inclined relative to the longitudinal axis of the anchor element.

This measure has the advantage that the slits can be formed in the anchor element in such a way that, by pulling the received suture in the proximal direction, said suture easily slides radially inward into the slit and remains there. A suture, once it has been received and tensioned, remains In the inclined slit of the anchor element. A secure hold of the suture is therefore guaranteed as long as the suture is held taut in the anchor element.

The anchor element, which is pushed onto the tool, can be secured captive on the tool with the aid of the taut suture. This permits reliable introduction of the anchor element into the bone.

In another embodiment, the inclination has an angle of approximately 10° to approximately 80°.

This measure has the advantage that the slit, in terms of its inclination, can be designed differently depending on the intended use and design of the anchor element.

This permits an inclination that is sufficiently steep to ensure that a received suture can still be held securely in the slit even if the tensioning is relaxed. These variations are possible by virtue of the jacket of thick material.

In another embodiment, the at least one slit has a rounded base.

This measure has the advantage that the suture can to a certain extent execute transverse movements in the slit, the possibility of fraying being ruled out in the rounded base.

In another embodiment, the rounded base has a curved course.

This measure has the advantage that, in a base with a curved course, the suture bears over a relatively long portion of the slit. This avoids local points of contact for the suture. In this way, tensile forces that arise are conveyed uniformly to the anchor element.

In another embodiment, the curved course is U-shaped.

This measure has the advantage that a U-shaped course of the curvature allows proximally directed forces on the suture to be conveyed optimally to the anchor element.

In another embodiment, the anchor element has several slits.

This measure has the advantage that several sutures can be received in the slits in the anchor element. In this way, tensile forces are distributed across different sutures and positions in the anchor element and are thereby reduced. For this reason, these tensile forces are conveyed more uniformly to the anchor element.

In another embodiment, the anchor element has two slits.

This measure has the advantage that two slits in the anchor element represent a very good compromise between the aforementioned advantages and the material removed from the anchor element.

In another embodiment, the slits are arranged diametrically in the anchor element.

This measure has the advantage that securing the anchor element on the tool with the aid of the sutures threaded into the slits takes place in a uniform manner. Forces that act on the anchor element during introduction are in this way distributed uniformly.

In addition, the anchor element and also the sutures are thus secured captive on the tool during introduction.

This measure also has the advantage that tensile loads acting on the inserted sutures are conveyed to the bone at opposite parts of the anchor element that has been introduced. This ensures uniform loading of the anchor element in a loading situation in the bone. Damage to the anchor element by locally delimited peak forces is thus ruled out.

The object is achieved, in another and second alternative, by an anchor element having an elongated body, said body having a central bore for receiving a tool for introducing said anchor element into said bone, said body having a proximal end section surrounding said central bore, said end section being designed as a jacket of considerable substance, wherein at least one approximately U-shaped channel being provided in said jacket of considerable substance, said at least one U-shaped channel opens in a proximal end face of said jacket of considerable substance, said at least one suture is received in said at least one U-shaped channel.

These measures have the advantage that a suture placed in the channel and subjected to tensile stress bears tightly and uniformly against a channel wall and thus ensures uniform distribution of the tensile force over a portion of the channel.

The anchor element can also be designed to meet different levels of tensile forces, by means of the channel being formed to a suitable depth in the anchor element. This is supported by the fact that the channel is located in the jacket of considerable substance and thus in a solid portion of the anchor element with a great wall thickness.

This embodiment further affords the advantage that the suture holder and the course of a central bore in the anchor element can be kept spatially separate from one another.

This allows the depth of the central bore to be adapted to differently designed anchor elements. This ensures an optimal depth of insertion of a tool into the anchor element.

It is additionally advantageous that a suture received in the proximal portion of the anchor element can be guided directly with its thread ends in the proximal direction toward the tool. The suture is thus guided away from the direct operating site and toward the tool, and interference with the operation is avoided. The suture received in the channel cannot fall out to the sides.

In another embodiment, the channel is widened in a funnel shape at the proximal end face.

This measure has the advantage of making it easier to insert the at least one suture into the channel of the anchor element.

In another embodiment, the anchor element has several U-shaped channels.

This measure has the advantage that several sutures can be received simultaneously in the anchor element. In this way, forces that arise are distributed between different sutures and positions in the anchor element and are thereby reduced. The anchor element is exposed to less material stress than is caused by a single substantial force that occurs locally.

Moreover, the increased number of suture ends allows an operating surgeon a more varied approach when securing a tendon or a ligament.

In another embodiment, the anchor element has two U-shaped channels.

This measure has the advantage that two channels in the anchor element represent a very good compromise between the aforementioned advantages and the material removed from the anchor element.

In another embodiment, the two U-shaped channels are arranged diametrically in the anchor element.

This measure has the advantage that securing the anchor element on the tool with the aid of the sutures threaded into the channels takes place in a uniform manner. Forces that act on the anchor element during introduction are in this way distributed uniformly.

In addition, the anchor element and also the sutures are thus secured captive on the tool during introduction.

This measure also has the advantage that tensile loads acting on the inserted sutures are conveyed to the bone at opposite parts of the anchor element that has been introduced. This ensures uniform loading of the anchor element in a loading situation in the bone. Damage to the anchor element by locally delimited peak forces is thus ruled out.

The object is achieved, in another and third alternative, by an anchor element having an elongated body, said body having a central bore for receiving a tool for introducing said anchor element into said bone, said body having a proximal end section surrounding said central bore, said end section being designed as a jacket of considerable substance, wherein at least one suture eyelet member projects in a proximal direction from said jacket of considerable substance, said suture eyelet member having an opening, said at least one suture is received in said eyelet member opening.

These measures have the advantage that a suture placed in the suture eyelet takes the forces acting on it and conveys them uniformly to the jacket of considerable substance of the anchor element.

A suture holder in the form of a suture eyelet of this design provides the suture with a suitably large contact surface. This contact surface extends within a part of the jacket of thick material of the anchor element. In this way, the suture takes the forces acting on it and conveys them to a solid portion of the anchor element.

The more elongate the suture eyelet in which the suture is received in the anchor element, the greater also is the contact surface of the suture. In this way, the forces acting on the suture are conveyed uniformly to the anchor element across a greater surface area, and locally concentrated forces are avoided. This protects the suture eyelet from being torn out or broken off.

This embodiment also has the advantage that the suture holder and the course of a central bore in the anchor element can be kept spatially separate from one another.

This allows the depth of the central bore to be adapted to different designs of anchor elements. In this way, an optimal depth of introduction of a tool into the anchor element is ensured. A tool pushed into the anchor element ensures reliable insertion of the anchor element into the bone.

In another embodiment, the suture eyelet member extends along a secant in the area of the jacket of considerable substance.

This measure has the advantage that the suture eyelet member extends across a long area and thus offers an inserted suture a suitably large surface area of contact. In this way, the forces acting on the suture are conveyed uniformly to the anchor element across a greater surface area, and locally concentrated forces are avoided.

The danger of the suture eyelet member being torn out is therefore minimized by the greater distribution of the forces in the suture eyelet.

In another embodiment, the at least one opening in the suture eyelet member is beveled.

This measure has the advantage of preventing fraying of the suture which, in accordance with its function, extends upward at the opening of the suture eyelet member. Damage or severing of the suture is thereby avoided.

In another embodiment, the at least one opening in the suture eyelet member has a curved course.

This measure has the advantage that the suture in a curved suture eyelet member is in contact uniformly along its entire course. This avoids individual, local contact points for the suture. In this way, tensile forces that arise are conveyed uniformly to the anchor element, and stressing of the anchor element is further reduced.

In another embodiment, the curved course is U-shaped.

This measure has the advantage that, when exposed to tensile stress, the suture arranged in a suture eyelet member extending in a U-shaped curve bears tightly and uniformly against a wall of the suture eyelet member. This permits a uniform distribution of the tensile force across the suture eyelet member. As has already been described, this represents an ideal case of optimal distribution of force to the anchor element.

In this way, the forces acting on the suture eyelet member are conveyed uniformly to the anchor element across a greater surface area, and locally concentrated forces are avoided. This protects the suture eyelet member from being torn out or broken away from the jacked of considerable substance.

In another embodiment, the anchor element has several suture eyelet members.

This measure has the advantage that the anchor element can receive several sutures through several suture eyelet members. Forces that arise are reduced into portions across a plurality of sutures and are in this way conveyed uniformly to different locations of the anchor element.

The anchor element experiences less material stress than is caused by a single substantial force that occurs locally.

Moreover, the increased number of suture ends allows an operating surgeon a more varied approach when securing a tendon or a ligament.

In another embodiment, the anchor element has two suture eyelet members.

This measure has the advantage of affording an optimal compromise between the number of suture eyelet members and the stress acting on the jacket of considerable substance.

In another embodiment, the two suture eyelet members are arranged diametrically in the anchor element.

This measure has the advantage that the openings of the suture eyelets are distributed uniformly across the proximal portion of the jacket. Securing the anchor element to the tool with the aid of the sutures threaded into the slits therefore also takes place with uniform orientation. In this way, forces that act on the anchor element during introduction are uniformly distributed.

It will be appreciated that the aforementioned features and the features still to be explained below can be used not only in the stated combination but also in other combinations or singly, without departing from the scope of the present invention.

### BRIEF DESCRIPTION OF THE DRAWINGS

The invention, in its three alternative forms, is described and explained in more detail below on the basis of selected illustrative embodiments and with reference to the attached drawings, in which:

FIG. 1 shows a side view of a first alternative of an anchor element,

FIG. 2 shows a section along the line II-II in FIG. 1,

FIG. 3 shows a side view of second alternative of an anchor element,

FIG. 4 shows a section along the line IV-IV in FIG. 3,

FIG. 5 shows a side view of third alternative of an anchor element,

FIG. 6 shows a view of the proximal end of the anchor element from FIG. 5,

FIG. 7 shows a side view of the anchor element from FIG. 1 and of a distal end of a tool which is intended to be inserted from proximal to distal into the anchor element,

FIG. 8 shows the assembly of anchor element and tool from FIG. 7 joined together, with inserted sutures, and

FIG. 9 shows a situation when introducing the assembly.

### DETAILED DESCRIPTION OF PREFERRED EMBODIMENTS

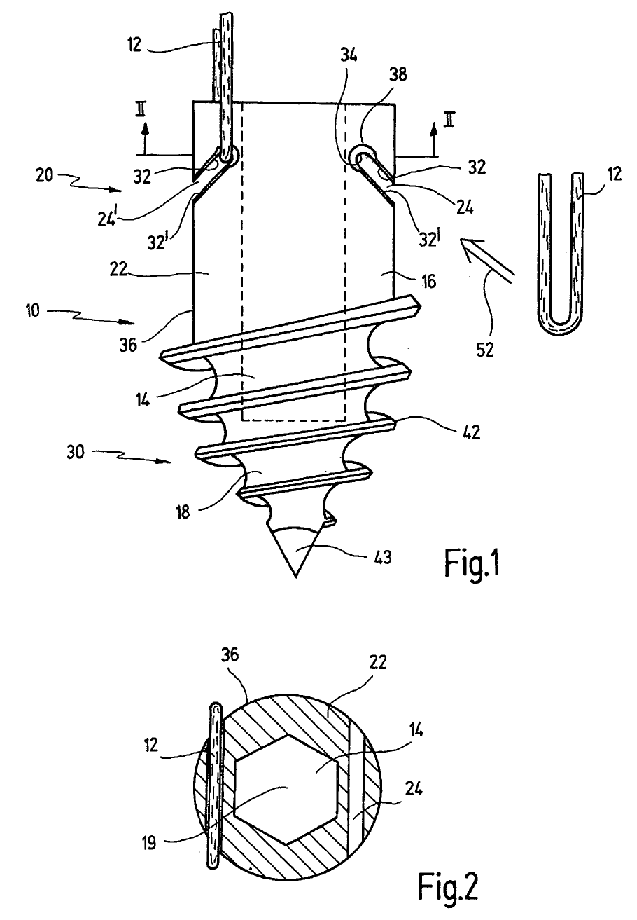

Surgical suture anchor elements according to the invention are shown in each of FIGS. 1, 3 and 5 and are respectively designated in their entirety by reference numbers 10, 10′ and 10″.

The anchor element 10 in FIGS. 1 and 2 has an elongate, approximately cylindrical body 16 in its proximal portion 20. This cylindrical body 16 merges into a taper 18 in a distal portion 30.

A self-tapping thread 42 extends in the distal direction along the taper 18. This self-tapping thread 42 runs out in a tip 43.

In its proximal portion 20, the anchor element 10 has a central bore 14. This central bore 14 is designed as a blind hole.

This central bore 14 extends axially along the central longitudinal axis 19 of the anchor element 10. It reaches deep into the anchor element 10 and, as is shown in FIG. 2, has a hexagonal cross section. The latter serves to receive a tool in a rotationally fixed manner.

The central bore 14 is surrounded about its circumference by a jacket 22 of considerable substance. The jacket 22 is a part of the anchor element 10 with most and thick material compared to the other parts of the anchor element 10.

Two slits 24 and 24′ are formed in this jacket 22 of thick material. They extend transversely with respect to the central longitudinal axis 19 of the anchor element 10. In doing so, they extend equally in the circumferential direction of the jacket 22 of thick material.

The slits 24 and 24′ each have flanks 32 and 32′, respectively. These flanks 32, 32′ extend in parallel into the anchor element 10 and join together at a rounded base 34.

The parallel flanks 32, 32′ of each slit 24, 24′ open laterally outward onto a radial outer face 36.

The flanks 32, 32′ and also the rounded base 34 are beveled at their transition to the radial outer face 36.

To securely receive the suture, the flanks 32, 32′ of the slits 24, 24′ are inclined in the distal direction, as seen radially outward from the base 34.

It will be seen clearly that a suture 12 which is inserted into the slits 24, 24′ and is held taut cannot escape distally out of the slit 24, 24′.

FIG. 2 illustrates how the central bore 14 is surrounded about its circumference by the jacket 22 of considerable substance. This jacket 22 of thick material has its material reduced only by the slits 24, 24′.

It will also be seen that the central bore 14 and the slits 24, 24′ are spatially separated. This means that the tool 44 is received in a manner unimpeded by a suture holder in the anchor element 10.

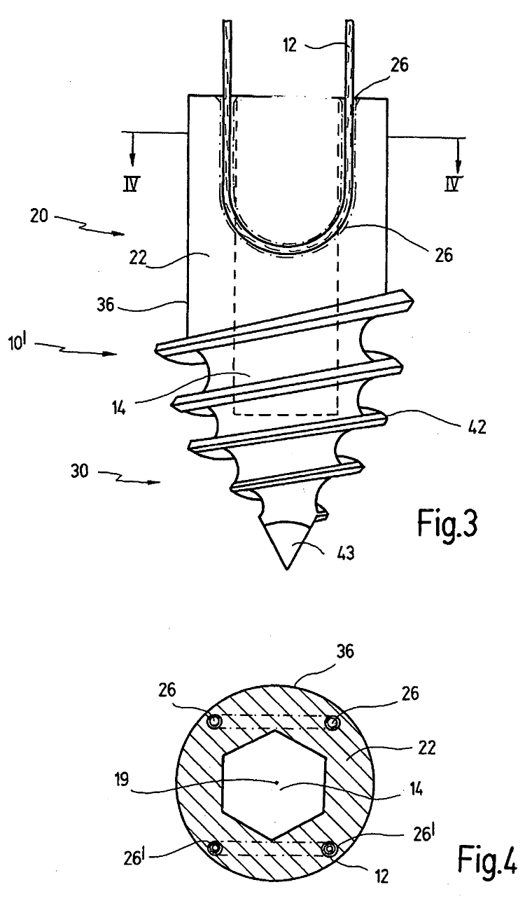

The anchor element 10′ shown in FIG. 3 also has an elongate, approximately cylindrical body in its proximal portion 20. The cylindrical body merges into a taper in a distal portion 30.

A self-tapping thread 42 extends in the distal direction along the taper 18. This self-tapping thread 42 runs out in a tip 43.

Along its entire length, the tip 43 also has a notch which supports the self-tapping thread 42 during screwing-in.

The anchor element 10′ also has a central bore 14 in its proximal portion 20, as is shown in FIG. 3. This central bore 14 is likewise designed as a blind hole.

As has already been described, the central bore 14 extends axially along the central longitudinal axis of the anchor element 10′. It again reaches deep into the anchor element 10′ and, as is shown in FIG. 4, has a hexagonal cross section. The latter serves to receive a tool in a rotationally fixed manner.

In this case too, the central bore 14 is surrounded about its circumference by a jacket 22 of considerable substance.

The jacket 22 of thick material has two openings at the proximal end face. They are each formed as a U-shaped channel 26 and 26′ which opens out in the proximal direction.

For easier introduction of a suture 12, these channels 26, 26′ are widened in a funnel shape at the proximal end.

The cross section in FIG. 4 illustrates how the central bore 14 is surrounded about its circumference by the jacket 22 of thick material. This jacket 22 of thick material has its material thickness reduced only by the channels 26, 26′.

It will also be seen that the central bore 14 is formed in the anchor element 10′ separate from the channels 26, 26′. This means that the tool is received in a manner unimpeded by a suture holder in the anchor element 10′.

When the anchor element 10′ is produced as an injection-molded part, the injection mold can contain, in the area of the channels 26 and 26′, a U-shaped wire which has the form of the channels 26 and 26′ and which is subsequently removed from the injection-molded part.

When the anchor element 10′ is produced as a metal part, it can be suitably preshaped as a metal-powder blank and then sintered.

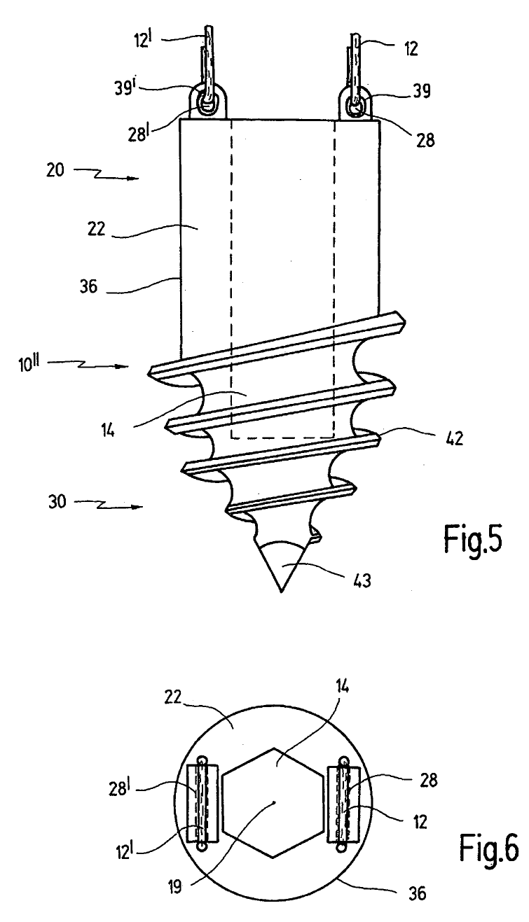

Another anchor element 10″ is shown in FIG. 5.

This anchor element 10″ also has an elongate, approximately cylindrical body in its proximal portion 20. The cylindrical body merges into a taper in a distal portion 30.

In this case too, a self-tapping thread 42 extends in the distal direction along the taper. This self-tapping thread 42 also runs out in a tip 43.

Along its entire length, the tip 43 also has a notch which supports the self-tapping thread 42 during screwing-in.

The anchor element 10″ also has a central bore 14 in its proximal portion 20, as is shown in FIG. 5. This central bore 14 is likewise designed as a blind hole.

As has already been described, the central bore 14 extends axially along the central longitudinal axis of the anchor element 10″. It reaches deep into the anchor element 10″ and, as is shown in FIG. 6, has a hexagonal cross section. The latter serves to receive a tool 44 in a rotationally fixed manner.

In this case too, in the proximal portion 20, the central bore 14 is surrounded about its circumference by a jacket 22 of considerable substance. This jacket 22 of thick material also gives this anchor element 10″ its outwardly cylindrical shape.

Two suture eyelet members 28 and 28′ project in the proximal direction from this jacket 22 of thick material. These suture eyelet members 28 and 28′ have beveled openings.

The suture eyelet members 28, 28′ extend along secants of the circular end face on the jacket 22 of thick material. A suture 12, 12′ is threaded respectively into these suture eyelet members 28, 28′. The taut sutures 12, 12′ point with their suture ends in the proximal direction. This is illustrated in FIGS. 5 and 6.

FIG. 6 again illustrates how, in this case too, the central bore 14 is surrounded about its circumference by the jacket 22 of thick material. As is shown here, however, this jacket 22 of thick material is completely unaffected by a material reduction.

It will also be seen here that the central bore 14 is formed in the anchor element 10″ separate from the suture eyelet members 28, 28′. This means that, in this case too, the tool 44 is received in a manner unimpeded by a suture holder in the anchor element 10″.

As can be seen from FIGS. 1, 3 and 5, the respective anchor element 10, 10′, 10″ has specific openings in the form of slits, channels and suture eyelets. Sutures 12, 12′ can be received in these in such a way that both free ends of the suture extend away from the anchor element 10, 10′, 10″ in the proximal direction. The sutures 12, 12′ are in each case arranged diametrically in the anchor element 10, 10′, 10″.

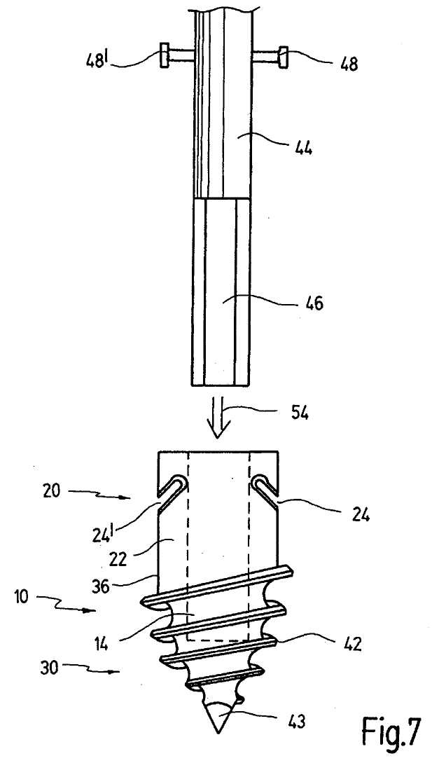

It will be seen from FIG. 7 that a tool 44 can be pushed from proximal to distal into the central bore 14 of the anchor element 10.

The tool 44 has a hexagonal distal end 46. The cross section of the distal end 46 corresponds to the cross section of the central bore 14. The distal end 46 can thus be pushed into the central bore 14 as indicated by an arrow 54 in FIG. 7.

The distal end 46 is thus received in a positive and rotationally fixed manner in the anchor element 10. The depth of insertion of the distal end 46 is adapted to the central bore 14. Obstruction by transversely extending sutures 12, 12′ is avoided in this arrangement.

As will also be seen from FIG. 7, pins 48, 48′ protrude radially from the tool 44. These pins 48, 48′ are arranged diametrically on the tool 44 and serve for the threading and fixing of the sutures 12, 12′.

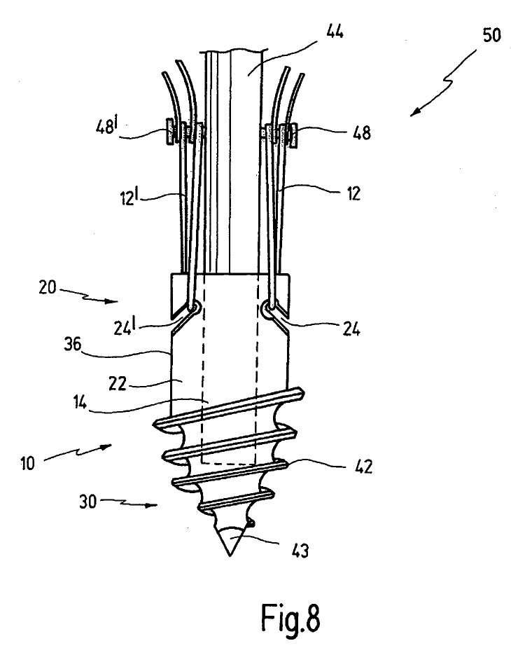

A tool 44 pushed fully into the anchor element 10 is shown in FIG. 8.

A suture 12, 12′ is now inserted into the respective slit 24, 24′, held taut, and fixed over the pins 48, 48′. In this way, the anchor element 10 is secured against slipping or loss during introduction.

This is supported by the hexagonal cross section of the central bore 14 of the respective anchor element 10. By this means, the tool 44 is received in a positive and rotationally fixed manner in the anchor element 10.

A complete tool 50 composed of anchor element 10, sutures 12, 12′ and tool 44 and used for introduction of an anchor element is shown in FIG. 8.

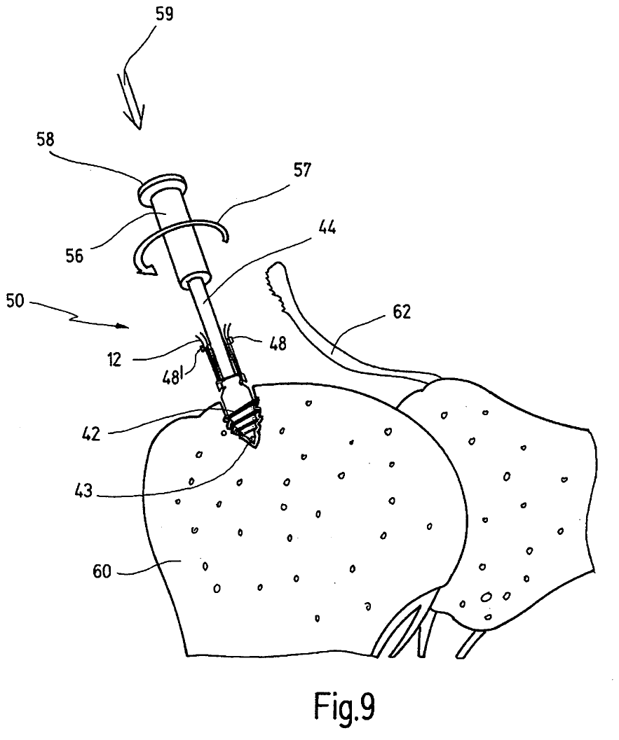

To introduce the anchor element 10, the complete tool 50, as shown in FIG. 9, is now initially placed with the tip 43 of the anchor element 10 at a certain position on the bone, for example a shoulder bone 60. To be able to turn the anchor element 10 into the shoulder bone 60, the tool 44 has a grip 56 at the proximal end. The turning of the tool 44 is indicated by an arrow 57 in FIG. 9. The self-tapping thread 42 bores its own way into the bone 60. The tip 43 supports the self-tapping thread 42.

Alternatively, the anchor element 10 can also at first be driven in linearly by a hammer blow to the tool 44, as is indicated by an arrow 59. Then, by turning the complete tool 50, the anchor element 10 is turned into the bone 60, this procedure being supported by the thread 42. During this procedure, the suture 12, 12′ is secured against displacement or twisting, since it is fixed on the pins 48, 48′.

After the anchor element 10 has been turned to the full extent into the shoulder bone 60, the tool 44 is withdrawn in the proximal direction. In doing this, the anchor element 10 remains in the bone 60. Using the protruding ends of the suture 12, 12′, it is now possible to fix a tendon 62 that has been torn from the shoulder bone 60.

This procedure can equally be carried out with the anchor elements 10′ and 10″.

If it is made of absorbable material, an anchor element 10, 10′, 10″ that has been introduced in this way so as to bear tightly on the bone substance can be gradually replaced by bone substance, such that the tendon 62 is then once again fixed in a manner true to nature.

What is claimed is:

1. A surgical suture anchor element for anchoring at least one suture in a bone, said at least one suture being provided for fixing at least one of a tendon, a ligament, a soft tissue at said bone,
said anchor element having an elongated body,
said body having a central bore for receiving a tool for introducing said anchor element into said bone, said body having a proximal end section surrounding said central bore, said end section being designed as a jacket of considerable substance,
wherein at least one slit is provided in said jacket of considerable substance,
said slit opens laterally to a radial outer face of said body,
said at least one slit extending in a circumferential direction within said jacket of considerable substance, and
said at least one suture is received within said at least one slit.

2. The surgical suture anchor element of claim 1, wherein said at least one slit has flanks extending in parallel.

3. The surgical suture anchor element of claim 1, wherein said at least one slit has flanks that diverge.

4. The surgical suture anchor element of claim 1, wherein said at least one slit has flanks which are beveled at edges thereof.

5. The surgical suture anchor element of claim 1, wherein said at least one slit is inclined relative to a longitudinal axis of said elongated body of said anchor element.

6. The surgical suture anchor element of claim 5, wherein said at least one slit is inclined relative to the longitudinal axis by an angle of approximately 10° to approximately 80°.

7. The surgical suture anchor element of claim 1, wherein said at least one slit has a rounded base.

8. The surgical suture anchor element of claim 7, wherein said rounded base has a curved course.

9. The surgical suture anchor element of claim 8, wherein said curved course together with two flanks of said slit is U-shaped.

10. The surgical suture anchor element of claim 1, wherein several slits are provided in said jacket of considerable substance.

11. The surgical suture anchor element of claim 10, wherein two slits are provided in said jacket of considerable substance.

12. The surgical suture anchor element of claim 11, wherein said two slits are arranged diametrically in said jacket of considerable substance.

13. A surgical suture anchor element for anchoring at least one suture in a bone, said at least one suture being provided for fixing at least one of a tendon, a ligament, a soft tissue at said bone,
said anchor element having an elongated body,
said body having a central bore for receiving a tool for introducing said anchor element into said bone, said body having a proximal end section surrounding said central bore, said end section being designed as a jacket of considerable substance,
wherein at least one approximately U-shaped channel being provided in said jacket of considerable substance, said at least one U-shaped channel opens in a proximal end face of said jacket of considerable substance,
said at least one suture is received in said at least one U-shaped channel.

14. The surgical suture anchor element of claim 13, wherein said channel widens in a funnel shape at said proximal end face.

15. The surgical suture anchor element of claim 13, wherein several U-shaped channels are provided within said jacket of considerable substance.

16. The surgical suture anchor element of claim 15, wherein two U-shaped channels are provided within said jacket of considerable substance.

17. The surgical suture anchor element of claim 16, wherein said two U-shaped channels are arranged diametrically in said jacket of considerable substance.

18. A surgical suture anchor element for anchoring at least one suture in a bone, said at least one suture being provided for fixing at least one of a tendon, a ligament, a soft tissue at said bone,
said anchor element having an elongated body,
said body having a central bore for receiving a tool for introducing said anchor element into said bone, said body having a proximal end section surrounding said central bore, said end section being designed as a jacket of considerable substance,
wherein at least one suture eyelet member projects in a proximal direction from said jacket of considerable substance, said suture eyelet member having an opening,
said at least one suture is received in said eyelet member opening.

19. The surgical suture anchor element of claim 18, wherein said suture eyelet member extends along a secant of said jacket of considerable substance.

20. The surgical suture anchor element of claim 18, wherein said opening in said suture eyelet element is beveled.

21. The surgical suture anchor element of claim 18, wherein said opening in said suture eyelet member has a curved course.

22. The surgical suture anchor element of claim 21, wherein said curved course is U-shaped.

23. The surgical suture anchor element of claim 18, wherein several suture eyelet members are provided at said jacket of considerable substance.

24. The surgical suture anchor element of claim 23, wherein two suture eyelet members project from said jacket of considerable substance.

25. The surgical suture anchor element of claim 24, wherein said two suture eyelet members are arranged diametrically at said jacket of considerable substance.
